(19) United States
(12) Patent Application Publication
(10) Pub. No.: US 2023/0256036 A1
(43) Pub. Date: Aug. 17, 2023

(54) SERPIN PRODUCTION

(71) Applicant: SOCIETE DES PRODUITS NESTLE S.A., Vevey (CH)

(72) Inventors: STEPHANE DUBOUX, St-Prex (CH); MICHIEL KLEEREBEZEM, Wageningen (NL); MIREILLE GOLLIARD, Lausanne 26 (CH)

(21) Appl. No.: 18/003,001

(22) PCT Filed: Jun. 25, 2021
(86) PCT No.: PCT/EP2021/067448

§ 371 Date: Dec. 22, 2022

(30) Foreign Application Priority Data
Jun. 26, 2020 (EP) 20182651.8

(51) Int. Cl. A61K 35/745; C12N 1/20; C12P 21/02; C07K 14/81; A61K 31/702

(52) U.S. Cl. CPC A61K 35/745; C12N 1/205; C12P 21/02; C07K 14/8121; A61K 31/702; C12N 2500/34

(57) ABSTRACT

Use of human milk oligosaccharide LNT, for increasing serpin protein production in Bifidobacterium longum subsp. longum.

### FIELD OF THE INVENTION

The present invention relates to bacteria expressing serpin, methods for increasing serpin production in bacteria and uses thereof in young mammalian subjects.

### BACKGROUND TO THE INVENTION

Serine protease inhibitors (serpin) are a superfamily of proteins found in eukaryotes (Gettins, 2002, Chemical reviews, 102(12), 4751-4804) and prokaryotes (Kantyka et al., Biochimie, 92(11), 1644-1656).

More recently, serpins have been reported in prokaryotes. In silico analysis revealed the presence of genes encoding serpin-like proteins in different Bifidobacterium species, particularly in bacteria of the species Bifidobacterium longum subsp longum. The protein encoded by B. longum subsp longum (named B. longum) NCC 2705 displayed similar antiprotease activity to those of human serpin (Ivanov et al 2006, Journal of Biological Chemistry, 281(25), 17246-17252).

B. longum NCC 2705 was deposited with the Institute Pasteur according to the Budapest Treaty on 29 Jan. 2001 receiving the deposit no. CNCM I-2618.

Bifidobacterial serpin was proposed to play an important role in the colonization of bifidobacteria by protecting them against host-derived proteases and providing them with a survival advantage in the competitive intestinal environment (Ivanov et al. 2006; Turroni et al. 2010).

In healthy, vaginally-delivered, breast-fed infant, Bifidobacteria form the basis of the microbiota in the infant gut. The amount of Bifidobacteria varies over the first months of life starting in average slightly below 10% at birth and increasing up to a peak of nearly 40% between 2.5 and 3 months.

Breast feeding also promotes intestinal barrier development which, together with bifidobacterial domination leads to enhanced absorption and therefore utilisation of ingested nutrition.

According to recent estimates and depending on the geographical location, B. longum subsp. longum can make up to 20% of the Bifidobacterium community in the intestine, which can constitute up to 4% of the overall microbiota in adults. At the same time, genomic studies have convincingly shown that Bifidobacteria present in the gut of breast-fed infants, such as Bifidobacterium longum, are specially equipped to utilize breast-milk oligosaccharides as nutrients. Bifidobacterium longum is also adapted to the conditions in the large intestine where energy harvest from slowly absorbable carbohydrates takes place (Turroni et al. 2018, Bifidobacteria and the infant gut: an example of co-evolution and natural selection. Cell 390 Mol Life Sci 75:103-118).

More and more evidence is emerging which suggests that the establishment of an appropriate intestinal microbiota early in life may, including population of B. longum subsp. longum, be significant in subsequent healthy development.

It is therefore clear that there is a need to provide a means to promote the rapid establishment of an appropriate intestinal microbiota in infants.

The serpin's capacity to inhibit the Human Neutrophil Elastase (HNE) (Ivanov et al. 2006) may also be involved in the immunomodulatory capacities of the strain (Riedel et al. 2006) as elastase is released by activated neutrophils at the sites of intestinal inflammation (Burg and Pillinger 2001). In line with this role in dampening innate immunity, serpin was recently demonstrated to play a key role in the anti-inflammatory effect of B. longum NCC 2705 in a mouse model of gluten sensitivity (McCarville et al., 2017, Appl. Envoron. Microbiol. Vol. 83, no. 19, e01323-17). Recently, the purified serpin from the NCC 2705 (CNCM I-2618) strain was reported to prevent enteric nerve activation in vitro, which potentially could reduce gastrointestinal pain in Irritable Bowel Syndrome (IBS) patients. (Buhner et al. 2018).

Additionally, there is a need to provide nutritional solution which could treat or prevent inflammatory conditions in the gut in infants.

### SUMMARY OF THE INVENTION

The present inventors have surprisingly found that LNT can increase the production of serpin when added to the growth medium of bacteria of the species Bifidobacterium longum subsp longum.

Accordingly, in a first aspect of the present invention, there is provided use of LNT, for increasing serpin production in Bifidobacterium longum subsp longum.

In another aspect of the present invention, there is provided a method of increasing serpin production in a bacteria of the species Bifidobacterium longum subsp longum, wherein said method comprises growing Bifidobacterium longum subsp longum in a culture medium, characterised in that said culture medium comprises LNT.

According to another aspect of the present invention, there is provided a bacteria of the species Bifidobacterium longum subsp longum produced by a method of growing the Bifidobacterium longum subsp longum in a culture medium, characterised in that said culture medium comprises LNT.

The Bifidobacterium longum subsp longum produced according to the present invention is associated with increased serpin protein levels relative to the same Bifidobacterium longum subsp longum strain grown in the absence of LNT.

According to the present invention, the Bifidobacterium longum subsp longum may be cultured in a medium comprising LNT, at a concentration of, for example, 0.02 to 5 wt %, preferably 0.05 to 2 wt %.

For example, the B. longum strain CNCM I-2618 may be cultured in a medium comprising LNT, at a concentration of 0.02 to 5 wt %, 0.05 to 2 wt %, 0.1 to 1.5 wt %, or about 0.5%.

According to another aspect of the present invention, there is provided a composition comprising a Bifidobacterium longum subsp longum produced according to the method described herein.

In one embodiment, the composition is a food, a medical food, a tube feed, or a nutritional composition.

In one embodiment, the food is selected from milk, yoghurt, curd, cheese, fermented milks, milk based fermented products, rice based products, milk based powders, and pet food.

In another embodiment, the nutritional composition is an infant formula, a growing up milk, a fortifier and/or supplement, for example a paediatric or maternal supplement.

According to another aspect of the present invention there is provided a Bifidobacterium longum subsp longum produced according to the method described herein, or a composition comprising said Bifidobacterium longum subsp longum, for use in promoting formation of bifidogenic intestinal microbiota in infants or children.

According to another aspect of the present invention there is provided a Bifidobacterium longum subsp longum produced according to the method described herein, or a composition comprising said Bifidobacterium longum subsp longum, for use in the treatment or prevention of gut inflammatory conditions in infants or children.

In one embodiment of the present invention, the gut inflammatory condition is a condition selected from the group consisting of enterocolitis, NEC (necrotizing enterocolitis), and inflammation associated to a gastro intestinal infection.

The Bifidobacterium longum subsp longum may be any Bifidobacterium longum subsp longum strain. In some preferred embodiments the Bifidobacterium longum subsp longum strain may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), or a combination thereof, in particular B. longum CNCM I-2618 (NCC 2705).

It will also be appreciated that LNT may also increase the production of serpin in Bifidobacterium longum subsp longum in vivo when LNT is administered in combination with the Bifidobacterium longum subsp longum.

Thus, according to another aspect of the present invention there is also provided a combination of (i) a Bifidobacterium longum subsp longum and (ii) LNT.

According to another aspect of the present invention there is also provided a combination of (i) a Bifidobacterium longum subsp longum and (ii) LNT, for use in promoting formation of bifidogenic intestinal microbiota in infants or children by increasing in situ serpin protein production.

According to another aspect of the present invention there is also provided a combination of (i) a Bifidobacterium longum subsp longum and (ii) LNT, for use in in the treatment or prevention of gut inflammatory conditions in infants or children.

In one embodiment, the treatment or prevention of gut inflammatory conditions in infants or children occurs by increasing in situ serpin protein production.

In one embodiment of the present invention, the gut inflammatory condition is a condition selected from the group consisting of enterocolitis, NEC (necrotizing enterocolitis), and inflammation associated to a gastro intestinal infection.

In one embodiment, the combination is a combination of B. longum strain CNCM I-2618 and LNT.

According to another aspect of the present invention there is also provided Bifidobacterium longum subsp longum for use in promoting formation of bifidogenic intestinal microbiota in infants or children by increasing serpin protein production, wherein the Bifidobacterium longum subsp longum is administered in combination with LNT.

According to another aspect of the present invention there is provided LNT for use in promoting formation of bifidogenic intestinal microbiota in infants or children by increasing serpin protein production, wherein the LNT, is administered in combination with Bifidobacterium longum subsp longum.

According to another aspect of the present invention there is also provided Bifidobacterium longum subsp longum for use in the treatment or prevention of gut inflammatory conditions in infants or children, wherein the Bifidobacterium longum subsp longum is administered in combination with LNT.

According to another aspect of the present invention there is provided LNT for use in the treatment or prevention of gut inflammatory conditions in infants or children, wherein the LNT, is administered in combination with Bifidobacterium longum subsp longum.

In some embodiments the Bifidobacterium longum subsp longum may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), or a combination thereof.

In some preferred embodiments, the Bifidobacterium longum subsp longum may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), or a combination thereof.

In some preferred embodiments, the Bifidobacterium longum subsp longum strain B. longum CNCM I-2618 (NCC 2705) is used.

### DESCRIPTION OF THE DRAWINGS

FIG. 1—Shows serpin protein levels measured in B. longum NCC 2705 incubated for 16 h on different human milk oligosaccharides, namely 2′-FL, LnNT, LNT3′-SL and DiFL.

FIG. 2—Shows serpin protein levels measured in a set of strains belonging to B. longum subsp. longum grown for 16 h on glucose or the human milk oligosaccharide LNT.

### DETAILED DESCRIPTION OF THE INVENTION

### Definitions

Within the context of the present invention the term “bifidogenic intestinal microbiota” means an intestinal microbiota which is dominated by Bifidobacteria such as Bifidobacterium breve, Bifidobacterium infantis, and Bifidobacterium longum, in particular Bifidobacterium longum.

The term “infant” means a child under the age of 12 months. The expression “child” means a between 12 months and seven years of age. The expression “young child” means a child aged between one and less than three years, also called toddler.

An “infant or young child born by C-section” means an infant or young child who was delivered by caesarean. It means that the infant or young child was not vaginally delivered.

An “infant or young child vaginally born” means an infant or young child who was vaginally delivered and not delivered by caesarean.

A “preterm” or “premature” means an infant or young child who was not born at term. Generally it refers to an infant or young child born prior 37 weeks of gestation.

An “infant having a low birth weight” means a new born having a body weight below 2500 g (5.5 pounds) either because of preterm birth or restricted fetal growth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

An “infant born small for gestational age (SGA)” means a baby with birth weights below the 10th percentile for babies of the same gestational age.

The expression “nutritional composition” means a composition which may nourish a subject. This nutritional composition is usually to be taken orally or intravenously, and it usually includes a lipid or fat source, a carbohydrate source and/or and a protein source. Non limiting examples of nutritional compositions are: infant formula, follow up formula, baby food, growing up milk, fortifier, paediatric supplements or infant cereal compositions.

In a particular embodiment the composition of the present invention is a hypoallergenic nutritional composition. The expression “hypoallergenic nutritional composition” means a nutritional composition which is unlikely to cause allergic reactions.

In a particular embodiment the composition or nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The term “fortifier” refers to liquid or solid nutritional compositions suitable for fortifying or mixing with human milk, infant formula, growing-up milk or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The term “nutritional supplement” refers to a product which is intended to supplement the general diet of a subject.

The term “paediatric supplement” refers to a product which is intended to supplement the general diet of a infant or a child.

The expression “weaning period” means the period during which the mother's milk is substituted by other food in the diet of an infant or young child.

The expressions “gastrointestinal tract”, “GI tract”, “GIT”, “gut” and “GUT” can be used interchangeably. The tract consists of the stomach and intestines, and is divided into the upper gastrointestinal tract and the lower gastrointestinal tract. It refers to the system (including digestive organs) responsible for consuming and digesting foodstuffs, absorbing nutrients, and expelling waste. The GI tract especially includes all digestive structures between the mouth and the anus. The upper gastrointestinal tract typically includes the oesophagus and the stomach. The lower gastrointestinal tract typically includes the small intestine and all of the large intestine (colon).

The expression “preventing and/or treating gastrointestinal inflammations” encompasses one or several of the following:
preventing gastrointestinal inflammations, i.e. inflammations of the gut
treating gastrointestinal inflammations, i.e. inflammations of the gut

Within the context of the present invention, the expression “in situ serpin production” refers to production of serpin protein in the gut of the subject, for example an infant or child, to whom the combination is administered.

### Composition

The composition of the present invention may be in the form of a food, a medical food, a tube feed, a nutritional composition, or a nutritional supplement.

In one embodiment, the food is selected from milk, yoghurt, curd, cheese, fermented milks, milk based fermented products, rice based products, milk based powders, infant formulae and pet food.

The composition may be in the form of a medical food. The term “medical food” as used herein refers to a food product specifically formulated for the dietary management of a medical disease or condition. The medical food may be administered under medical supervision. The medical food may be for oral ingestion or tube feeding.

The composition may be in the form of a tube feed. The term “tube feed” refers to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes), or a feeding tube placed directly into the abdomen of a subject (such as gastrostomy, gastrojejunostomy, or jejunostomy feeding tube).

In another embodiment, the nutritional composition or synthetic nutritional composition is selected is selected in the group consisting of: infant formula, follow up formula, baby food, growing up milk, fortifier, paediatric supplements and infant cereal compositions.

It is clear to those skilled in the art that an ideal dose will depend on the subject to be treated, its health condition, sex, age, or weight, for example, and the route of administration. The dose to be ideally used will consequently vary but can be determined easily by those of skill in the art.

However, generally, it is preferred if the composition of the present invention comprises between 10⁶ and 10¹⁰ cfu and/or between 10⁶ and 10¹⁰ cells of Bifidobacterium longum subsp longum per daily dose. It may also comprise between 10⁶ and 10¹¹ cfu and/or between 10⁶ and 10¹¹ cells of Bifidobacterium longum subsp longum per g of the dry weight of the composition.

Bifidobacterium longum

The Bifidobacterium longum may be any Bifidobacterium longum subsp longum strain. In some embodiments the Bifidobacterium longum subsp longum strain may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain NCIMB 8810, Bifidobacterium longum subsp longum strain CNCM I-2618 (NCC 2705), Bifidobacterium longum subsp longum strain CNCM I-2170, Bifidobacterium longum subsp longum strain ATCC 15707 (T), Bifidobacterium longum subsp longum strain CNCM I-103, Bifidobacterium longum subsp longum strain CNCM I-2334, Bifidobacterium longum subsp longum strain CNCM I-3864, Bifidobacterium longum subsp longum strain CNCM I-3853, or a combination thereof.

In another embodiment, the Bifidobacterium longum subsp longum strain may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain NCIMB 8810, Bifidobacterium longum subsp longum strain CNCM I-2618 (NCC 2705, Bifidobacterium longum subsp longum strain ATCC 15707 (T), or a combination thereof.

The strains have been deposited in the depositary institution indicated in the table below (Table 1), and have received the following date of deposit and accession number:

TABLE 1

| | Depositary | Accession | Date of |
|---|---|---|---|
| # | institution | number | deposit |
| 1 | CNCM | I-2618 | Jan. 29, 2001 |
| 2 | ATCC | 15707 | <1990 |
| 3 | CNCM | I-2169 | Mar. 15, 1999 |
| 4 | NCIMB | 8810 | Oct. 01, 1956 |
| 5 | CNCM | I-2171 | Mar. 15, 1999 |
| 6 | ATCC | 15708 | <1990 |
| 7 | DSM | 20097 | <1990 |
| 8 | NCIMB | 8809 | Oct. 01, 1956 |
| 9 | CNCM | I-2170 | Mar. 15, 1999 |
| 10 | CNCM | I-103 | Oct. 29, 1979 |
| 11 | CNCM | I-2334 | Oct. 12, 1999 |
| 1 | CNCM | I-2618 | Jan. 29, 2001 |
| 12 | CNCM | I-3864 | Nov. 15, 2007 |
| 13 | CNCM | I-3853 | Oct. 16, 2007 |

CNCM refers to Collection nationale de cultures de micro-organismes, Institut Pasteur, 28, rue du Dr Roux, F-75724 Paris Cedex 15, France. ATCC refers to American Type Culture Collection 10801 University Blvd., Manassas, Va. 20110-2209, U.S.A. DSM refers to Leibniz Institute DSMZ-German Collection of Microorganisms and Cell Cultures, Inhoffenstr. 7B, D-38124 Braunschweig, Germany. NCIMB refers to NCIMB Ltd, Ferguson Building, Craibstone Estate, Buckburn, Aberdeen AB21 9YA, Scotland.

Strains 1, 2, 5, 9-13 have been deposited by Nestec S.A., avenue Nestlé 55, 1800 Vevey, Switzerland. Since then, Nestec S.A. has merged into Societe des Produits Nestlé S.A. Accordingly, Societe des Produits Nestlé S.A. is the successor in title of Nestec S.A., under article 2(ix) of the Budapest Treaty. All other strains are commercially available.

In some preferred embodiments, the Bifidobacterium longum subsp longum may be selected from Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), or a combination thereof.

In some preferred embodiments, the Bifidobacterium longum subsp longum strain B. longum CNCM I-2618 (NCC 2705) is used.

### LNT

The present inventors have surprisingly found that LNT can increase the production of serpin in bacteria of the species Bifidobacterium longum subsp longum.

Suitable sources of LNT are commercially available, and include for example Glycom.

The Bifidobacterium longum subsp longum may be cultured in a medium comprising LNT, at a concentration of, for example, 0.02 to 5 wt %. For example, the Bifidobacterium longum subsp longum may be cultured in a medium comprising galactose or GOS, or mixtures thereof, at a concentration 0.02 to 5 wt %, 0.05 to 2 wt %, 0.1 to 1.5 wt %, or about 0.5 wt %.

LNT may be added to a conventional culture medium comprising up to 8 wt %, preferably up to 6 wt %, for example up to 4 wt %, of another sugar suitable to sustain B. longum growth, such as, but not limited to, glucose. The inventors have surprisingly found that LNT can induce production of serpin in Bifidobacterium longum subsp longum even when glucose is present, but only when the glucose is present at levels allowing its depletion during fermentation. Preferably the culture medium at the end of the fermentation contains less than 0.4 wt % glucose, such as from 0 wt % to 0.3 wt % glucose, for example from 0.02 wt % to 0.4 wt %, or from about 0.05 wt % to about 0.3 wt %. Conventional culture mediums suitable for growth of B. longum are well known to the person skilled in the art.

In one embodiment, the Bifidobacterium longum subsp longum may be cultured in a medium comprising LNT at a concentration of, 0.05 to 2 wt %, 0.1 to 1.5 wt %, or about 0.5 wt %, optionally in the presence of glucose at a concentration enabling its depletion until the end of the fermentation. Preferably the culture medium at the end of the fermentation contains less than 0.4 wt % glucose, such as from 0 wt % to 0.3 wt % glucose. If glucose is present, the culture medium may contain, at the end of fermentation, for example, 0.02 wt % to 0.4 wt %, or about 0.05 wt % to about 0.3 wt % glucose.

### Process for Producing a Culture Powder

Strains belonging to the species B. longum are grown in anaerobic conditions. Fermentation methods under anaerobic conditions are commonly known. The skilled person is able to identify suitable components of the fermentation medium and to adjust fermentation conditions based on his general knowledge, depending on the microorganism to be grown. The fermentation medium typically comprises
a nitrogen source such as yeast extract,
a carbon source such as a sugar,
various growth factors (e.g minerals, vitamins etc.) required by the microorganism and
water.

A non-limiting example of a typical growth medium for B. longum is MRS (De Man, Rogosa and Sharpe) medium, supplemented with 0.05% of cysteine (MRSc).

The fermentation is preferably carried out in two steps, a starter fermentation being carried out prior to the main fermentation step. The fermentation medium can be different for the starter and the main fermentation or may be identical.

The second step of the process is the concentration of the biomass. This can also be carried out using methods known to the person skilled in the art, such as for example centrifugation or filtration. The total solid content of the biomass after concentration is preferably comprised between 10 and 35 wt %, preferably between 14 and 35 wt %, based on the total dry weight of the biomass (i.e. of the total amount of fermentation medium and produced microorganism).

Optionally, the concentration may be preceded or combined with a washing step to remove residues of the fermentation medium and/or compounds produced during fermentation. For example, washing may be performed by concentrating biomass, re-suspending the concentrated biomass in a buffer, such as a phosphate buffer, or a similar composition and re-concentrating the biomass.

For example, the process described in WO2017/001590, which is entirely incorporated by reference, can be applied.

### Combination

In one aspect of the present invention, there is provided a combination of (i) a Bifidobacterium longum subsp longum and (ii) LNT.

As used herein, the term “combination” refers to the combined administration of Bifidobacterium longum subsp longum and LNT, wherein the Bifidobacterium longum subsp longum and LNT may be administered simultaneously or sequentially.

As used herein, the term “simultaneous” or “simultaneously” is used to mean that the two agents are administered concurrently, i.e. at the same time.

The term “sequential” or “sequentially” is used to mean that the two agents are administered one after the other, wherein either the Bifidobacterium longum subsp longum or the galactose or GOS, or the combination thereof, may be administered first.

The agents may be administered either as separate formulations or as a single combined formulation.

When the compounds are co-formulated, i.e. in the same composition or formulation, they can only be administered simultaneously. When the compounds are formulated in separate compositions or formulations, they can be administered simultaneously or sequentially. Simultaneous administration of the agents in the same formulation or in separate formulations can also be described as the co- or joint administration of the two compounds.

In one embodiment, Bifidobacterium longum subsp longum and LNT are in admixture. In another embodiment, the Bifidobacterium longum subsp longum and LNT, are present in the form of a kit comprising a preparation of the two agents and, optionally, instructions for the simultaneous or sequential administration of the preparations to a subject in need thereof.

### Gut Inflammatory Disease

The Bifidobacterium longum subsp longum strains produced according to the present invention, or a composition comprising the same, may be for use in the treatment or prevention of gastro intestinal inflammatory conditions.

The gastrointestinal inflammations relate to inflammations involving the gastrointestinal tract. Similarly, there can be inflammations of the upper gastrointestinal tract or of the lower gastrointestinal tract. Examples of gastrointestinal inflammations are enterocolitis and NEC (necrotizing enterocolitis). The gastrointestinal infections may also be associated with a gastrointestinal inflammation.

Enterocolitis is an inflammation of the digestive tract, involving the small intestine and the colon. Common clinical manifestations of enterocolitis are frequent diarrheal defecations, with or without nausea, vomiting, abdominal pain, fever, chills, alteration of general condition. General manifestations are given by the dissemination of the infectious agent or its toxins throughout the body, or most frequently by significant losses of water and minerals, the consequence of diarrhea and vomiting.

Necrotizing enterocolitis (NEC) is a medical condition primarily seen in premature infants, where portions of the bowel undergo necrosis (tissue death). It occurs postnatally and it is the second most common cause of mortality in premature infants. Initial symptoms include feeding intolerance, increased gastric residuals, abdominal distension and bloody stools. The symptoms may progress rapidly to abdominal discoloration with important gut necrosis, intestinal perforation, peritonitis, systemic hypotension requiring intensive medical support, need of a surgical intervention, and sometimes death.

For example the Bifidobacterium longum subsp longum produced according to the present invention, or a composition comprising the same, or a combination according to the present invention may be for use in the treatment or prevention of a gastro intestinal inflammatory and inflammation associated to a gastro intestinal infections.

### Administration

The Bifidobacterium longum subsp longum or composition described herein are preferably administered enterally.

Enteral administration may be oral, gastric, and/or rectal.

In general terms, administration of the combination or composition described herein may, for example, be by an oral route or another route into the gastro-intestinal tract, for example the administration may be by tube feeding.

In an alternative embodiment administration of the combination or composition described herein may be topical administration.

The subject may be a young mammal such as a human, canine, feline, equine, caprine, bovine, ovine, porcine, cervine and primates. Preferably the subject is a young human.

In one embodiment of the present invention, the subject is a human infant or child. In another embodiment, the subject is an infant.

In one embodiment, the subject is the subject is an infant (up to 12 months of age) or a young child aged from 1 to 3 years of age).

In another embodiment, the subject is the subject is a child above 3 years and below 8 years of age.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1—B. longum NCC 2705 Serpin Induction by Human Milk Oligosaccharides

B. longum strain CNCM I-2618 (NCC 2705) was grown in Biolector (growth conditions—anaerobic, 37° C.) in MRS+5 mM L-cysteine (MRSc) base without sugar, to which different commercially available Human Milk Oligosaccharides (HMOs) were added at a concentration of 0.5 wt %.

48-well microtiter plate with pH sensor and dissolved oxygen (DO) sensor were used to culture the strains in Biolector (m2p-labs Aachen, Germany). It was continuously shaken to prevent bacteria aggregation for 16 h. Cultures were harvested by centrifugation and supernatant was removed. Pellet was resuspended in PBS supplemented with halt protease inhibitor (Sigma) and lysed using glassbeads. Lysate containing both soluble and insoluble material was then collected. Total protein content was measured using Pierce BCA kit (Thermofisher) and serpin protein concentration was determined using ELISA.

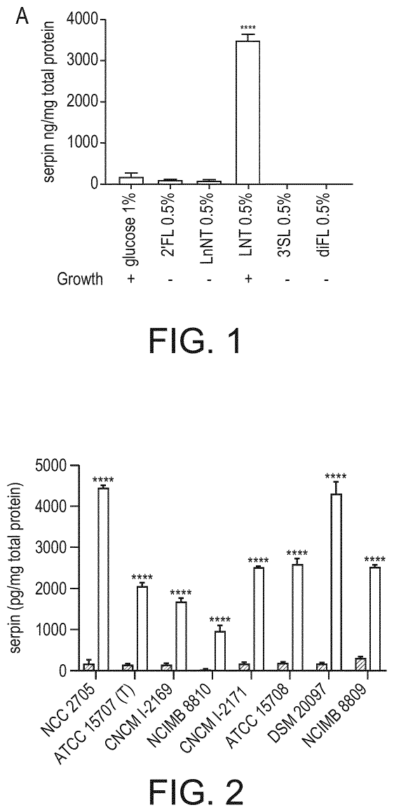

Out of all the tested HMOs, Lacto-N-tetraose (LNT) was the only one supporting the growth of B. longum NCC 2705. As shown in FIG. 1, LNT did not only support growth of the bacteria, but did as well significantly increase the levels of serpin protein in B. longum NCC 2705.

### Example 2—B. longum Subsp. longum Serpin Induction by LNT

The serpin encoding gene and its surrounding is highly conserved within the B. longum subsp. longum species. Strains of B. longum subsp. longum were selected to represent the entire span of the genetic phylogenetic tree (Table 2). All strains were cultured in Biolector (according to example 1) in a MRSc base without sugar, to which 0.5% glucose or 0.5% of LNT was added. Cultures were grown for 16 h and harvested. Obtained pellets were further analyzed for total and serpin protein content (see example 1).

TABLE 2
list of B. longum subsp. longum strains
tested and the homology of their
serpin gene to BL0108 (B. longum
NCC 2705 serpin encoding gene).

| | % ID to BL0108 |
|---|---|
| Strain nº | (NCC2705 serpin) |
| NCC 2705 | 100.00 |
| (CNCM I-2618) | |
| ATCC 15707 (T) | 99.78 |
| CNCM I-2171 | 99.79 |
| ATCC 15708 | 99.57 |
| DSM 20097 | 97.42 |
| NCIMB 8809 | 99.79 |
| CNCM I-2169 | 99.79 |
| NCIMB 8810 | 99.78 |

All strains of B. longum subsp. longum were able to grow on LNT as sole carbohydrate source. As shown in FIGS. 2, serpin protein levels were increased in all B. longum subsp. longum strains in presence of LNT, meaning that the induction capacity of LNT is conserved in all tested strains belonging to B. longum subsp. longum.

1-4. (canceled)

5. A method for increasing serpin protein levels in Bifidobacterium longum subsp longum wherein said method comprises growing Bifidobacterium longum subsp longum in a culture medium, characterised in that said culture medium LNT.

6. A method according to claim 5 wherein the culture medium comprises LNT at a concentration of 0.02 to 2 wt %.

7. A method according to claim 5 wherein the Bifidobacterium longum subsp longum is selected from the group consisting of Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), and a combination thereof.

8. Bifidobacterium longum subsp longum produced by a method of growing the Bifidobacterium longum subsp longum in a culture medium, wherein said culture medium comprises LNT.

9. Bifidobacterium longum subsp longum produced by the method of claim 8 wherein the culture medium comprises LNT at a concentration of 0.02 to 2 wt %.

10. Bifidobacterium longum subsp longum produced by the method of claim 8 wherein the Bifidobacterium longum subsp longum is selected from the group consisting of Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), Bifidobacterium longum subsp longum strain CNCM I-103, Bifidobacterium longum subsp longum strain CNCM I-2334, Bifidobacterium longum subsp longum strain CNCM I-3864, Bifidobacterium longum subsp longum strain CNCM I-3853, and a combination thereof.

11-13. (canceled)

14. A method for use in promoting formation of bifidogenic intestinal microbiota in infants or children by increasing in situ serpin protein production comprising administering a combination of (i) Bifidobacterium longum subsp longum and (ii) LNT to the infant or child.

15-16. (canceled)

17. A method according to claim 14 wherein the Bifidobacterium longum subsp longum is selected from the group consisting of Bifidobacterium longum subsp longum strain CNCM I-2169, Bifidobacterium longum subsp longum strain CNCM I-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM I-2618 (NCC 2705), Bifidobacterium longum subsp longum strain CNCM I-2170, Bifidobacterium longum subsp longum strain ATCC 15707 (T), Bifidobacterium longum subsp longum strain CNCM I-103, Bifidobacterium longum subsp longum strain CNCM I-2334, Bifidobacterium longum subsp longum strain CNCM I-3864, Bifidobacterium longum subsp longum strain CNCM I-3853, and a combination thereof.
